(19) United States
(12) Patent Application Publication
(10) Pub. No.: US 2005/0059880 A1
(43) Pub. Date: Mar. 17, 2005

(54) ECG driven image reconstruction for cardiac imaging

(76) Inventors: Sanjay George Mathias, Waukesha, WI (US); Kevin Scott Kreger, Milwaukee, WI (US); Thomas Leo Valent, Burlington, WI (US)

Correspondence Address: John S. Beulick;Armstrong Teasdale LLP, Suite 2600, One Metropolitan Square, St. Louis, MO, 63102, US

(21) Appl. No.: 10/660,216

(22) Filed: Sep. 11, 2003

(51) Int. Cl. A61B 5/05

(52) U.S. Cl. 600/413

(57) ABSTRACT

A method for generating an image of a heart at a selected cardiac phase is described. The method includes acquiring a first electrocardiogram (ECG) of the heart at a first phase, introducing a time delay into the first ECG to generate a phase-delayed ECG of the heart at the first phase, and using the first ECG and the phase-delayed ECG to generate an image of the heart.

### BACKGROUND OF THE INVENTION

This invention relates generally to magnetic resonance imaging (MRI), and more particularly to cardiac imaging using a MRI system.

The dynamic nature of a heart, and a desired temporal and spatial resolution for a reliable diagnosis, makes cardiac imaging a challenging task for MRI technology. Specifically, as the MRI system is scanning the heart, the heart continues to beat and move, and data is collected at varying cardiac phases. Since the data cannot be acquired instantaneously, so that the cardiac phase of the heart is known for each data set, electrocardiograph (ECG) data is collected to correlate, or ‘tag’, the MRI data with cardiac phase information. The ECG waveform represents the electrical activity of the heart and is correlated into the mechanical motion of the heart. The ECG waveform includes several identification points, P, Q, R, S, and T referred to herein as the QRS complex, which are used to provide cardiac phase information.

When the ECG is acquired using the MRI imaging system, the ECG includes noise generated by the static and dynamic magnetic field of the MRI system. In some known MRI imaging systems, the noise is strong enough to introduce inaccuracy in the detection of the peak of the ECG's QRS complex. The noise may result in the MRI system not identifying the QRS peaks, a false triggering on other parts of the ECG waveform, or time-related inaccuracies, i.e. jitter, in the detection of the QRS peaks. Therefore an inability to accurately determine the cardiac phase using the QRS complex of the ECG signal can reduce image quality.

### BRIEF DESCRIPTION OF THE INVENTION

In one aspect, a method for generating an image of a heart at a selected cardiac phase is provided. The method includes acquiring a first electrocardiogram (ECG) of the heart at a first phase, introducing a time delay into the first ECG to generate a phase-delayed ECG of the heart at the first phase, and using the first ECG and the phase-delayed ECG to generate an image of the heart.

In another aspect, a method for generating an image of a heart at a selected cardiac phase using an MRI imaging system is provided. The method includes acquiring a first electrocardiogram (ECG) of the heart at a first phase, acquiring a second electrocardiogram (ECG) of the heart at the first phase, and using the first ECG and the second ECG to generate an image of the heart.

In yet another aspect, a method for generating an image of a heart at a selected cardiac phase is provided. The method includes acquiring a first electrocardiogram (ECG) of the heart at a first phase, acquiring a first plethysmograph signal of the heart at a first phase, and using the first ECG and the first plethysmograph signal to generate an image of the heart.

In still another aspect, a magnetic resonance imaging (MRI) system is provided. The MRI system includes a radio frequency (RF) coil assembly for imaging a subject volume and a computer coupled to said RF coil. The computer is configured to acquire a first electrocardiogram (ECG) of the heart at a first phase, introduce a time delay into the first ECG to generate a phase-delayed ECG of the heart at the first phase, and use the first ECG and the phase-delayed ECG to generate an image of the heart.

In another aspect, a computer program embodied on a computer readable medium for controlling a medical imaging system is provided. The computer program is configured to acquire a first ECG of the heart at a first phase, acquire a second ECG of the heart at the first phase, and use the first ECG and the second ECG to generate an image of the heart.

### BRIEF DESCRIPTION OF THE DRAWINGS

FIG. 1 is a block schematic diagram of an exemplary Magnetic Resonance Imaging (MRI) system.

FIG. 2 is an exemplary method for acquiring an image of a heart at a selected cardiac phase.

FIG. 3 is a block schematic diagram of a control system that can be used with the MRI system shown in FIG. 1.

### DETAILED DESCRIPTION OF THE INVENTION

As used herein, an element or step recited in the singular and proceeded with the word “a” or “an” should be understood as not excluding plural said elements or steps, unless such exclusion is explicitly recited. Furthermore, references to “one embodiment” of the present invention are not intended to be interpreted as excluding the existence of additional embodiments that also incorporate the recited features.

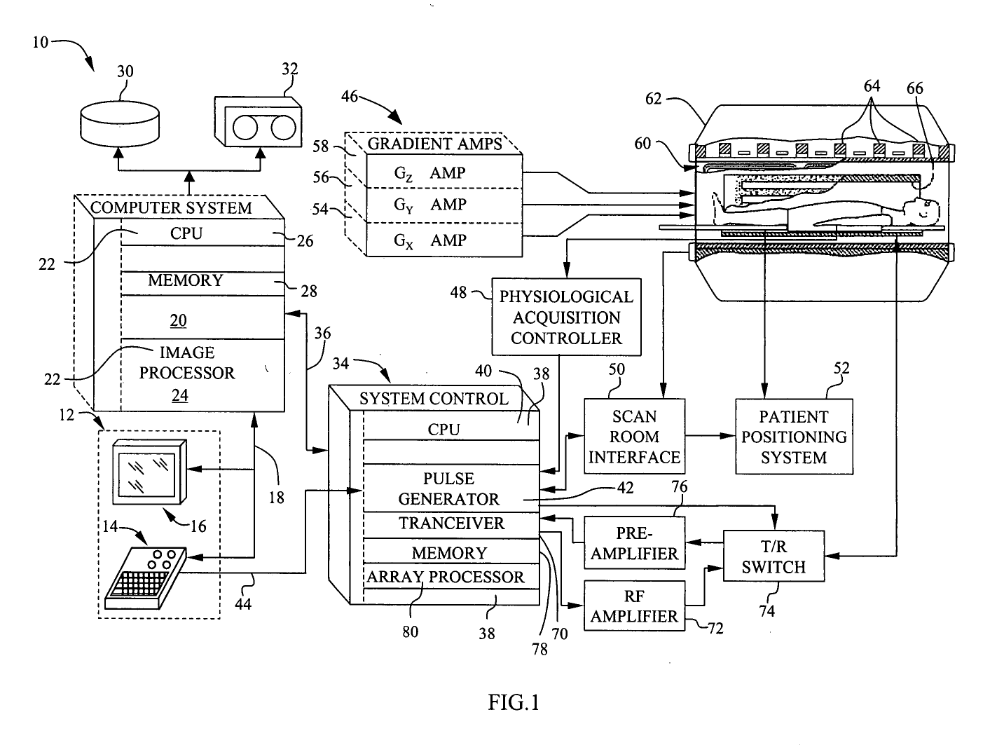

FIG. 1 is a block diagram of an embodiment of a magnetic resonance imaging (MRI) system 10 in which the herein described systems and methods are implemented. MRI system 10 includes an operator console 12 which includes a keyboard and control panel 14 and a display 16. Operator console 12 communicates through a link 18 with a separate computer system 20 thereby enabling an operator to control the production and display of images on screen 16. Computer system 20 includes a plurality of modules 22 which communicate with each other through a backplane. In the exemplary embodiment, modules 22 include an image processor module 24, a CPU module 26 and a memory module 28, also referred to herein as a frame buffer for storing image data arrays. Computer system 20 is linked to a disk storage 30 and a tape drive 32 to facilitate storing image data and programs. Computer system 20 is communicates with a separate system control 34 through a high speed serial link 36.

System control 34 includes a plurality of modules 38 electrically coupled using a backplane (not shown). In the exemplary embodiment, modules 38 include a CPU module 40 and a pulse generator module 42 that is electrically coupled to operator console 12 using a serial link 44. Link 44 facilitates transmitting and receiving commands between operator console 12 and system command 34 thereby allowing the operator to input a scan sequence that MRI system 10 is to perform. Pulse generator module 42 operates the system components to carry out the desired scan sequence, and generates data which indicative of the timing, strength and shape of the RF pulses which are to be produced, and the timing of and length of a data acquisition window. Pulse generator module 42 is electrically coupled to a gradient amplifier system 46 and provides gradient amplifier system 46 with a signal indicative of the timing and shape of the gradient pulses to be produced during the scan. Pulse generator module 42 is also configured to receive patient data from a physiological acquisition controller 48. In the exemplary embodiment, physiological acquisition controller 48 is configured to receive inputs from a plurality of sensors indicative of a patients physiological condition such as, but not limited to, ECG signals from electrodes attached to the patient. Pulse generator module 42 is electrically coupled to a scan room interface circuit 50 which is configured to receive signals from various sensors indicative of the patient condition and the magnet system. Scan room interface circuit 50 is also configured to transmit command signals such as, but not limited to, a command signal to move the patient to a desired position, to a patient positioning system 52.

The gradient waveforms produced by pulse generator module 42 are input to gradient amplifier system 46 that includes a G_{X} amplifier 54, a G_{Y} amplifier 56, and a G_{Z} amplifier 58. Amplifiers 54, 56, and 58 each excite a corresponding gradient coil in gradient coil assembly 60 to generate a plurality of magnetic field gradients used for position encoding acquired signals. In the exemplary embodiment, gradient coil assembly 60 includes a magnet assembly 62 that includes a polarizing magnet 64 and a whole-body RF coil 66.

In use, a transceiver module 70 positioned in system control 34 generates a plurality of electrical pulses which are amplified by an RF amplifier 72 that is electrically coupled to RF coil 66 using a transmit/receive switch 74. The resulting signals radiated by the excited nuclei in the patient are sensed by RF coil 66 and transmitted to a preamplifier 76 through transmit/receive switch 74. The amplified NMR (nuclear magnetic resonance) signals are then demodulated, filtered, and digitized in a receiver section of transceiver 70. Transmit/receive switch 74 is controlled by a signal from pulse generator module 42 to electrically connect RF amplifier 72 to coil 66 during the transmit mode and to connect preamplifier 76 during the receive mode. Transmit/receive switch 74 also enables a separate RF coil (for example, a surface coil) to be used in either the transmit or receive mode.

The NMR signals received by RF coil 66 are digitized by transceiver module 70 and transferred to a memory module 78 in system control 34. When the scan is completed and an array of raw k-space data has been acquired in the memory module 78. The raw k-space data is rearranged into separate k-space data arrays for each cardiac phase image to be reconstructed, and each of these is input to an array processor 80 configured to Fourier transform the data into an array of image data. This image data is transmitted through serial link 36 to computer system 20 where it is stored in disk memory 30. In response to commands received from operator console 12, this image data may be archived on tape drive 32, or it may be further processed by image processor 24 and transmitted to operator console 12 and presented on display 16.

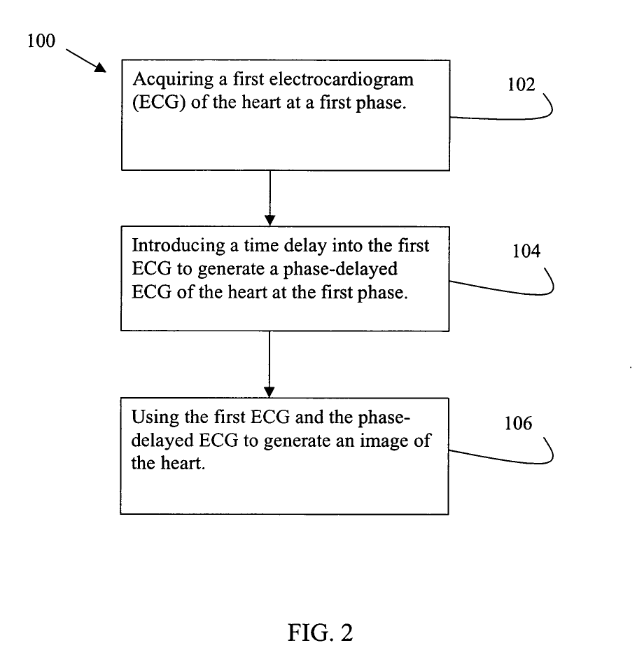

FIG. 2 is a method 100 for generating an image of a heart at a selected cardiac phase. Method 100 includes acquiring 102 a first electrocardiogram (ECG) of the heart at a first phase, introducing 104 a time delay into the first ECG to generate a phase-delayed ECG of the heart at the first phase, and using 106 the first ECG and the phase-delayed ECG to generate an image of the heart.

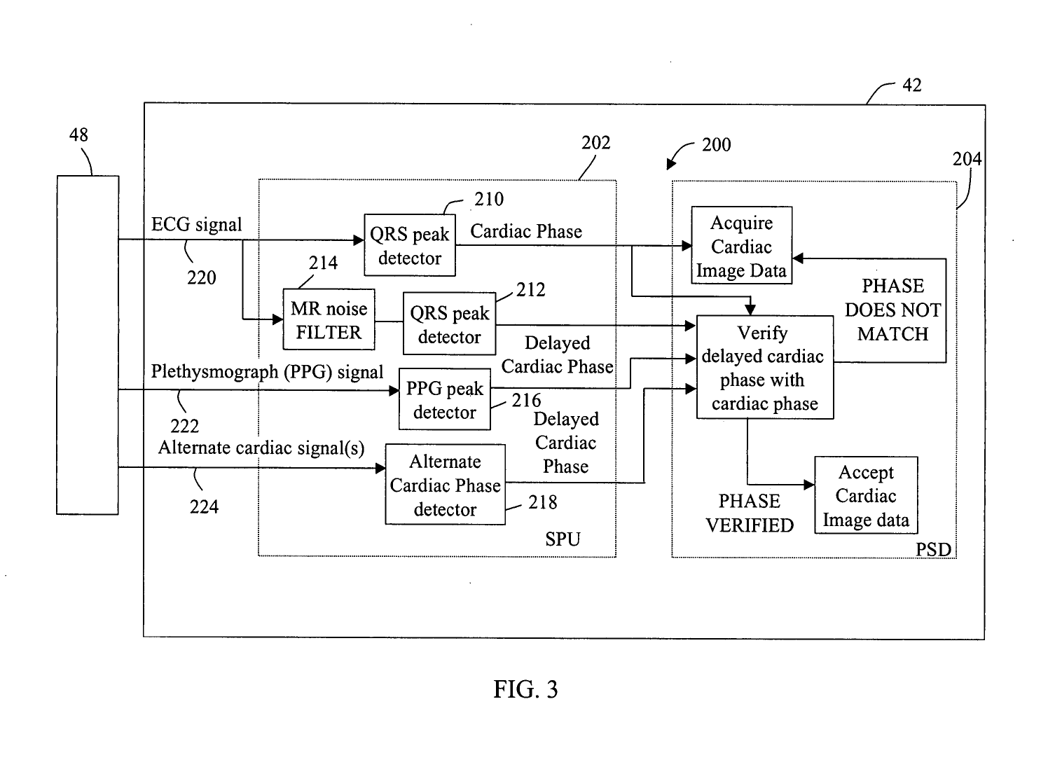

FIG. 3 is a schematic illustration of a control system 200 configured to acquire cardiac images that can be used with magnetic resonance imaging (MRI) system 10 shown in FIG. 1, and the method shown in FIG. 2. Control system 200 includes a Cardiac Signal Processing Unit (SPU) 202 and a Pulse Sequence Descriptor (PSD) 204. In the exemplary embodiment, SPU 202 and PSD 204 are software modules configured to run on pulse generator 42 and thereby control image acquisition. In another exemplary embodiment, the functions of SPU 202 and PSD 204 are implemented on dedicated hardware such as, but not limited to, an Application Specific Integrated Circuit (ASIC) or a digital signal processor (DSP).

SPU 202 includes a first QRS peak detector 210, a second QRS peak detector 212, a MRI noise filter 214, a plethysmograph (PPG) peak detector 216, and an alternate cardiac phase detector 218.

In use, a first ECG signal 220 is acquired by physiological acquisition controller 48 and input to pulse generator 42. First ECG signal 220 is then input to SPU 202 and MRI filter 214. A non-delayed output of QRS peak detector 210 is then input to PSD 204. In the exemplary embodiment, the output of QRS peak detector 210 includes cardiac phase information which is then input to PSD 204 to control image acquisition. Additionally, first ECG signal 220 is filtered using MRI filter 214. Filtering first ECG signal 220 facilitates generating more accurate phase information while also introducing a time delay into the filtered output of MRI noise filter 214. The output of MRI noise filter 214 is then input to a second QRS peak detector to generate delayed cardiac phase information which is then input to PSD 204.

As shown in FIG. 3, and in an exemplary embodiment, PSD 204 receives the non-delayed output from QRS peak detector 210 and the delayed output from QRS peak detector 212 to acquire an image of the heart. More specifically, if the delayed input and the non-delayed input received by PSD 204 approximately match, i.e., include phase information that is approximately equivalent, PSD 204 accepts the phase inputs from both from QRS peak detector 210 and QRS peak detector 212 and initiates system 10 to generate an image of the heart using the acquired cardiac phase information. Alternatively, if the delayed input and the non-delayed input received by PSD 204 do not approximately match, i.e., do not include phase information that is approximately equivalent, PSD 204 rejects the phase inputs from both from QRS peak detector 210 and QRS peak detector 212 and re-initiates system 10 to re-acquire cardiac information of the heart. Once PSD 204 has accepted the cardiac information received from QRS peak detector 210 and QRS peak detector 212, the cardiac phase information is used to generate an image of the heart.

In another exemplary embodiment, a PPG signal 222 is acquired by physiological acquisition controller 48 and input to pulse generator 42. In use, PPG signal 222 is input to PPG peak detector 216. The cardiac phase delayed output from PPG peak detector 216 is then input to PSD 220. If the delayed input, i.e. PPG peak detector 216 output, and the non-delayed input, i.e., QRS peak detector 210 output, received by PSD 204 approximately match, i.e., include phase information that is approximately equivalent, PSD 204 accepts the phase inputs from both from QRS peak detector 210 and PPG peak detector 216 and an image of the heart is generated using the acquired cardiac phase information. As an example, the phase information is approximately equivalent if phase of the delayed input is within plus or minus 10 milli-seconds of phase of the non-delayed input. In one embodiment, if the delayed input and the non-delayed input received by PSD 204 do not approximately match, i.e. do not include phase information that is approximately equivalent, PSD 204 rejects the phase inputs from both from QRS peak detector 210 and PPG peak detector 216 and re-initiates system 10 to re-acquire cardiac information of the heart. In another embodiment, if the delayed input and the non-delayed input received by PSD 204 do not approximately match, i.e. do not include phase information that is approximately equivalent, PSD 204 used the rejected phase inputs from both from QRS peak detector 210 and PPG peak detector 216 to extrapolate a correct position of ECG phase based on a known delay.

In yet another exemplary embodiment, a second cardiac signal 224 is acquired by physiological acquisition controller 48 and input to pulse generator 42. In use, second cardiac signal 224 is input to alternate cardiac phase detector 218. The cardiac phase delayed output from alternate cardiac phase detector 218 is then input to PSD 220. If the delayed input, i.e. alternate cardiac phase detector 218, and the non-delayed input, i.e. QRS peak detector 210 output, received by PSD 204 approximately match, i.e. include phase information that is approximately equivalent, PSD 204 accepts the phase inputs from both from QRS peak detector 210 and alternate cardiac phase detector 218 and an image of the heart is generated using the acquired cardiac phase information. In one embodiment, if the delayed input and the non-delayed input received by PSD 204 do not approximately match, i.e. do not include phase information that is approximately equivalent, PSD 204 rejects the phase inputs from both from QRS peak detector 210 and alternate cardiac phase detector 218 and re-initiates system 10 to re-acquire cardiac information of the heart. In another embodiment, if the delayed input and the non-delayed input received by PSD 204 do not approximately match, i.e. do not include phase information that is approximately equivalent, PSD 204 used the rejected phase inputs from both from QRS peak detector 210 and alternate cardiac phase detector 218 to extrapolate a correct position of ECG phase based on a known delay.

The methods and system described herein facilitate providing minimally delayed accurate phase information. For example, using either or both of these delayed cardiac signals, the “noisy” cardiac phase information can be reinforced to verify the correct phase information. Additionally, PSD 204 accepts the MRI cardiac image information only if PSD 204 determines that the phase delayed cardiac phase approximately matches the non-delayed cardiac phase, otherwise the information is rejected and new cardiac information is acquired or alternatively a corrected position of the ECG phase based on a known delay is extrapolated. Accordingly, the methods and system described herein can be utilized with a plurality of methods of monitoring heart activity including, but not limited to, Plethysmographs, Mechanical/Vibrational Sensors, and other electrical signals that are strongly filtered and/or delayed.

While the invention has been described in terms of various specific embodiments, those skilled in the art will recognize that the invention can be practiced with modification within the spirit and scope of the claims.

1. A method for generating an image of a heart at a selected cardiac phase, said method comprising:
acquiring a first electrocardiogram (ECG) of the heart at a first phase;
introducing a time delay into the first ECG to generate a phase-delayed ECG of the heart at the first phase; and
using the first ECG and the phase-delayed ECG to generate an image of the heart.

2. A method in accordance with claim 1 wherein said using the first ECG and the phase-delayed ECG to generate an image of the heart comprises using the first ECG and the phase-delayed ECG to generate an MRI image of the heart.

3. A method in accordance with claim 1 wherein said introducing a time delay into the first ECG comprises filtering the first ECG to introduce the time delay.

4. A method in accordance with claim 1 further comprising:
receiving at a pulse sequence descriptor (PSD) the first ECG and the phase-delayed ECG; and
using the PSD to determine if the first ECG and the phase-delayed ECG comprise the same approximate phase information.

5. A method in accordance with claim 4 further comprising:
rejecting the first ECG and the phase-delayed ECG based on the phase information included in the first ECG and the phase-delayed ECG; and
re-initializing an MRI system to re-acquire cardiac information of the heart.

6. A method in accordance with claim 4 further comprising:
rejecting the first ECG and the phase-delayed ECG based on the phase information included in the first ECG and the phase-delayed ECG; and
extrapolating a cardiac phase based on the phase information included in the first ECG and the phase-delayed ECG.

7. A method in accordance with claim 4 further comprising:
accepting the first ECG and the phase-delayed ECG; and
generating an image of the heart using the first ECG and the phase-delayed ECG.

8. A method for generating an image of a heart at a selected cardiac phase using an MRI imaging system, said method comprising:
acquiring a first electrocardiogram (ECG) of the heart at a first phase;
acquiring a second electrocardiogram (ECG) of the heart at the first phase; and
using the first ECG and the second ECG to generate an image of the heart.

9. A method in accordance with claim 8 further comprising:
receiving at a pulse sequence descriptor (PSD) the first ECG and the second ECG; and
determining if the first ECG and the second ECG comprise the same approximate phase information.

10. A method in accordance with claim 9 further comprising:
rejecting the first ECG and the second ECG based on the phase information in the first ECG and the second ECG; and
re-initializing an MRI system to re-acquire cardiac information of the heart.

11. A method in accordance with claim 9 further comprising:
accepting the first ECG and the phase-delayed ECG based on the phase information in the first ECG and the phase-delayed ECG; and
generating an image of the heart using the first ECG and the phase-delayed ECG.

12. A method for generating an image of a heart at a selected cardiac phase, said method comprising:
acquiring a first electrocardiogram (ECG) of the heart at a first phase;
acquiring a first plethysmograph signal of the heart at a first phase; and
using the first ECG and the first plethysmograph signal to generate an image of the heart.

13. A method in accordance with claim 12 wherein said acquiring a first electrocardiogram (ECG) of the heart at a first phase comprises acquiring a first plethysmograph signal of the heart at a first phase using a magnetic resonance imaging (MRI) system.

14. A method in accordance with claim 12 further comprising:
receiving at a pulse sequence descriptor (PSD) the first ECG and the first plethysmograph signal; and
determining if the first ECG and the first plethysmograph signal comprise the same approximate phase information.

15. A method in accordance with claim 14 further comprising:
rejecting the first ECG and the first plethysmograph signal based on the phase information in the first ECG and the first plethysmograph signal; and
re-initializing the MRI system to re-acquire cardiac information of the heart.

16. A method in accordance with claim 14 further comprising:
accepting the first ECG and the first plethysmograph signal based on the phase information in the first ECG and the first plethysmograph signal; and
generating an image of the heart using the first ECG and the first plethysmograph signal.

17. A magnetic resonance imaging (MRI) system comprising:
a radio frequency (RF) coil assembly for imaging a subject volume; and
a computer coupled to said RF coil, said computer configured to:
acquire a first electrocardiogram (ECG) of the heart at a first phase;
introduce a time delay into the first ECG to generate a phase-delayed ECG of the heart at the first phase; and
use the first ECG and the phase-delayed ECG to generate an image of the heart.

18. An MRI system in accordance with claim 17 wherein said computer is further configured to filter the first ECG to introduce the time delay.

19. An MRI system in accordance with claim 17 wherein said computer is further configured to:
receive at a pulse sequence descriptor (PSD) the first ECG and the phase-delayed ECG; and
determine if the first ECG and the phase-delayed ECG have the same approximate phase information.

20. An MRI system in accordance with claim 17 wherein said computer is further configured to:
reject the first ECG and the phase-delayed ECG based on the phase information included in the first ECG and the phase-delayed ECG; and
re-initiate the MRI system to re-acquire cardiac information of the heart.

21. An MRI system in accordance with claim 17 wherein said computer is further configured to:
accept the first ECG and the phase-delayed ECG; and
generate an image of the heart using the first ECG and the phase-delayed ECG.

22. A computer program embodied on a computer readable medium for controlling a medical imaging system, said program configured to:
acquire a first electrocardiogram (ECG) of the heart at a first phase;
acquire a second electrocardiogram (ECG) of the heart at the first phase; and
use the first ECG and the second ECG to generate an image of the heart.

23. A computer program in accordance with claim 22 wherein said program further configured to:
receive at a pulse sequence descriptor (PSD) the first ECG and the second ECG; and
determine if the first ECG and the second ECG comprise the same approximate phase information.

24. A computer program in accordance with claim 22 wherein said program further configured to:
reject the first ECG and the second ECG based on the phase information in the first ECG and the second ECG; and
re-initiate the MRI system to re-acquire cardiac information of the heart.

25. A computer program in accordance with claim 22 wherein said program further configured to:
accept the first ECG and the phase-delayed ECG based on the phase information in the first ECG and the phase-delayed ECG; and
generate an image of the heart using the first ECG and the phase-delayed ECG.
